SEQUENCE LISTING (18 sequences)

SEQ ID NO: 1 - protein, 457 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHKVIRGRYLVALGHAYHPEEFVCSQCGKVLEEGGFFEEKGAIFCPPCYDVRYAPSCAKCKKKITGEIMHALKMTWHVHCFTCAACKTPIRNRAFYMEEGVPYCERDYEKMFGTKCHGCDFKIDAGDRFLEALGFSWHDTCFVCAICQINLEGKTFYSKKDRPLCKSHAFSHV

SEQ ID NO: 2 - protein, 423 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKVQTPDKQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHKVIRGRYLVALGHAYHPEEFVCSQCGKVLEEGGFFEEKGAIFCPPCYDVRYAPSCAKCKKKITGEIMHALKMTWHVHCFTCAACKTPIRNRAFYMEEGVPYCERDYEKMFGTKCHGCDFKIDAGDRFLEALGFSWHDTCFVCAICQINLEGKTFYSKKDRPLCKSHAFSHV

SEQ ID NO: 3 - protein, 153 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGCRPLTNSRSDRWSQMPASSG

SEQ ID NO: 4 - protein, 223 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPP

SEQ ID NO: 5 - protein, 53 aa, Homo sapiens
CHGCDFKIDAGDRFLEALGFSWHDTCFVCAICQINLEGKTFYSKKDRPLCKSH

SEQ ID NO: 6 - protein, 51 aa, Homo sapiens
CAKCKKKITGEIMHALKMTWHVHCFTCAACKTPIRNRAFYMEEGVPYCERD

SEQ ID NO: 7 - protein, 10 aa, Homo sapiens
GAPPPADSAP

SEQ ID NO: 8 - protein, 9 aa, Artificial
  a TAT domain
RKKRRQRRR

SEQ ID NO: 9 - protein, 16 aa, Artificial
  a PTD domain
RQIKIWFQNRRMKWKK

SEQ ID NO: 10 - protein, 14 aa, Artificial
  a PTD domain
GRKKRRQRRRPPQC

SEQ ID NO: 11 - protein, 26 aa, Artificial
  a PTD domain
GWTLNSAGYLLKINLKALAALAKKIL

SEQ ID NO: 12 - protein, 9 aa, Artificial
  a PTD domain
RRRRRRRRR

SEQ ID NO: 13 - protein, 12 aa, Artificial
  a PTD domain
PIRRRKKLRRLK

SEQ ID NO: 14 - protein, 12 aa, Artificial
  a PTD domain
RRQRRTSKLMKR

SEQ ID NO: 15 - protein, 12 aa, Artificial
  a PTD domain
SRRKRQRSNMRI

SEQ ID NO: 16 - protein, 12 aa, Artificial
  a PTD domain
SFHQFARATLAS

SEQ ID NO: 17 - protein, 12 aa, Artificial
  a PTD domain
DPATNPGPHFPR

SEQ ID NO: 18 - protein, 12 aa, Artificial
  a PTD domain
TLPSPLALLTVH

(19) United States
(12) Patent Application Publication
(10) Pub. No.: US 2009/0110637 A1
(43) Pub. Date: Apr. 30, 2009

(54) LMP and Regulation of Tissue Growth

(75) Inventors: Jeffrey C. Marx, Germantown, TN (US); William F. McKay, Memphis, TN (US); Susan J. Drapeau, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/924,960

(22) Filed: Oct. 26, 2007

(51) Int. Cl. A61K 38/00; A61F 2/00; A61K 49/00; A61P 43/00; C12N 5/02

(52) U.S. Cl. 424/9.1; 424/423; 435/377; 514/12; 514/4

(57) ABSTRACT

Novel methods are provided for changing cell phenotype, comprising a method of changing a phenotype of a target cell comprising: increasing an amount of an amino acid sequence in a source cell, wherein the source cell is located within a volume of a media and wherein the amino acid sequence is selected from the group consisting of LMP-1 protein, an LMP-2 protein, an LMP-3 protein, an LMP-1s protein, SEQ ID NO: 5, SEQ ID NO: 6, SEQ ID NO: 7 or combination thereof; collecting at least a portion of the volume of the media; and contacting the target cell with at least the portion of the media.

### FIELD OF THE INVENTION

The present invention relates to the application of LIM mineralization proteins (LMPs) for the regulation of tissue growth.

### BACKGROUND OF THE INVENTION

LMP is a pluripotent molecule, which regulates or influences a number of biological processes. The different splice variants of LMP are expected to have different biological functions in mammals. This invention involves the role they play in the growth, differentiation, and/or regeneration of various tissues. For example, some form of LMP is expressed not only in bone, but also in muscle, tendons, ligaments, spinal cord, peripheral nerves, and cartilage. One isoform of LMP, LMP-1, has been detected in adult rat kidney, heart, brain, and lung. Boden et al., Endocrinology 139(12): 5125-5134 (1998).

LMP-1 contains an N-terminal PDZ domain and three C-terminal LIM domains/motifs. David et al. LIM domains: multiple roles as adapters and functional modifiers in protein interactions. Trends Genet 1998; 14:156-62. The LMP proteins enhance tissue mineralization in mammalian cells grown in vitro. When produced in mammals, LMP also induces tissue formation in vivo.

LMP-1 is a highly conserved intracellular regulator protein, which has been shown to increase proteoglycan production by upregulating multiple bone morphogenetic proteins (BMPs). See S. T. Yoon et al., ISSLS Prize Winner: LMP-1 Upregulates Intervertebral Disc Cell Production of Proteoglycans and BMPs In Vitro and In Vivo, 29 SPINE 2603-11 (2004).

The LIM domain is a cysteine-rich structural motif composed of two special zinc fingers that are joined by a 2-amino acid spacer. Some proteins have only LIM domains, while others contain a variety of additional functional domains. LIM proteins form a diverse group, which includes transcription factors and cytoskeletal proteins. The primary role of LIM domains appears to be in mediating protein-protein interactions, through the formation of dimers with identical or different LIM domains, or by binding distinct proteins.

In LIM homeodomain proteins, that is, proteins having both LIM domains and a homeodomain sequence, the LIM domains function as negative regulatory elements. LIM homeodomain proteins are involved in the control of cell lineage determination and the regulation of differentiation, although LIM-only proteins may have similar roles. LIM-only proteins are also implicated in the control of cell proliferation since several genes encoding such proteins are associated with oncogenic chromosome translocations.

Human protein LMP-1 has been disclosed previously. LMP-1 contains an N-terminal PDZ domain and three C-terminal LIM domains. Several isoforms of the LMP protein have been characterized: LMP-1, as discussed above, LMP-2 (which contains a 119 base pair deletion between bp 325 and 444, and a 17 bp insertion at bp 444, compared to LMP-1), LMP-3 (which does not have a deletion but has a 17 bp insertion at bp 444, thus resulting in a shift in a reading frame and a stop codon at bp 505), and truncated (short) version of LMP-1, termed LMP-1s, containing the N-terminal 223 amino acids of the full length hLMP-1, while maintaining osteoinductive activity. Liu et al, J. Bone Miner. Res. 17(3): 406-414 (2002), incorporated herein by reference in its entirety.

It has also been previously found that LMP is capable of inducing expression of genes encoding proteins which are secreted into the interstitial fluid and involved in autocrine and paracrine cell signalling. Suitable examples of such proteins include, without limitation, members of the bone morphogenic protein family, such as BMP-2 and BMP-7. Yoon et al., (2004). Bone morphogenic proteins have been shown to be involved with embryonic dorsal-ventral patterning, limb bud development, and fracture repair in adult animals. Hogan, Genes & Develop., 10, 1580 (1996). This group of transforming growth factor-beta super-family secreted proteins has a spectrum of activities in a variety of cell types at different stages of differentiation; differences in physiological activity between these closely related molecules have not been clarified. D. M. Kingsley, Trends Genet., 10, 16 (1994).

Research into LMPs to stimulate proteoglycan and BMP upregulation has previously only focused on intervertebral disc cells or bone marrow cells, specifically dealing with bone growth. The development of methods utilizing LMPs to promote growth of other tissues has been lacking.

### SUMMARY OF THE INVENTION

The instant invention fulfills this and other needs by providing, in one aspect, a method of regulating tissue growth and development without drawbacks associated with “conventional” in vivo gene therapy. In addition, rather than administering several biological agents (e.g., growth factors, hormones, etc), the invention provides a method of coordinated delivery of these biological agents thus allowing for a more natural healing process by allowing for the administration of anabolic and catabolic proteins to the targeted cells or tissues.

In one aspect, the invention provides a method of changing the phenotype of a target cell by increasing an amount of an amino acid sequence which is at least 70% identical in amino acid sequence of that encoding an LMP protein or a fragment thereof in a source cell, wherein the source cell is located within a volume of a media; collecting at least a portion of the volume of the media, and then contacting the target cell with at least a portion of the media. In different embodiments, the step of increasing an amount of an amino acid sequence which is at least 70% identical in amino acid sequence to that encoding an LMP protein or a fragment thereof is achieved by introducing to the source cell the amino acid sequence encoding an LMP protein or a fragment thereof or a nucleic acid sequence encoding such amino acid sequence encoding an LMP protein or a fragment thereof. In different embodiments, the LMP protein is an LMP-1 protein, an LMP-2 protein, LMP-3 protein, an LMP-1s protein, SEQ ID NO: 5, SEQ ID NO: 6, SEQ ID NO: 7, or any combination thereof.

In different embodiments, the source cell and the target cell are independently selected from the group consisting of kidney cells, neural cells, cardiac cells, smooth muscle cells, striated muscle cells, osteoblasts, osteoclasts, cartilage cells, endothelial cells, dental pulp cells, ligament cells, tendon cells, nucleus pulposus cells, annulus fibrosis cells, and any combination thereof.

In one embodiment, the target cell is located in vitro and later introduced into the patient by an injection or via an implant. In another embodiment, the target cell is located in the patient's body, and at least the portion of the volume of the media is delivered to the target cell via an implant or by an injection. In different embodiments, at least the portion of the volume of the media is isolated or concentrated prior to the delivery, or at least the portion of the volume of the media is delivered directly.

After being contacted with at least the portion of the media, the target cell changes at least one of its characteristics. The characteristic may be selected from morphology, electrical activity, contractility, migration, attachment, division rate, or gene expression pattern.

Accordingly, in another aspect, the invention provides a method of increasing the production of a target cell protein in a target cell comprising: (a) contacting a source cell with a polypeptide selected from the group consisting of an LMP-1 protein, an LMP-2 protein, an LMP-3 protein, an LMP-1s protein, SEQ ID NO: 5, SEQ ID NO: 6, SEQ ID NO: 7 or a combination thereof, (b) collecting a portion of the volume of the media of the source cell, and (c) contacting the target cell with the portion of the volume of the media of the source cell. In different embodiments, the target cell protein is selected from the group consisting of a kidney cell specific protein, a neural cell specific protein, a cardiac cell specific protein, a smooth muscle cell specific protein, a striated muscle cell specific protein, an osteoblast specific protein, an osteoclast specific protein, a cartilage cell specific protein, an endothelial cell specific protein, a dental pulp cell specific protein, a ligament cell specific protein, a tendon cells specific protein, a nucleus pulposus cell specific protein, and an annulus fibrosis cell specific protein.

In another aspect, the invention provides a method of generating an organ cell from a stem cell comprising (a) contacting a source cell with a polypeptide selected from the group consisting of an LMP-1 protein, an LMP-2 protein, an LMP-3 protein, an LMP-1s protein, SEQ ID NO: 5, SEQ ID NO: 6, SEQ ID NO: 7 or a combination thereof; (b) collecting a portion of the volume of the media of the source cell; and (c) contacting the stem cell with the portion of the volume of the media of the source cell. In different exemplary embodiments, the organ is a kidney, a nervous system, a heart, a smooth muscle, a striated muscle, a bone, a cartilage, a blood vessel, a tooth, a ligament, a tendon, or a disc.

### DETAILED DESCRIPTION OF PREFERRED EMBODIMENTS

The present invention describes novel compositions and methods for inducing tissue formation by using media from cultured cells with an increased amount of the amino acid sequence which is at least 70% identical to an amino acid sequence encoding an LMP protein or a fragment thereof.

For the purposes of better explaining the instant invention, the following non-limiting definitions are provided:

The phrase “changing gene expression pattern” refers to expressing genes which were not expressed previously; and also to significantly upregulating, downregulating or silencing expression of genes which are currently expressed.

The term “gene” refers to a DNA sequence that comprises control and coding sequences necessary for the production of a polypeptide or its precursor. The polypeptide can be encoded by a full length coding sequence (either genomic DNA or cDNA) or by any portion of the coding sequence so long as the desired activity is retained. In some aspects, the term “gene” also refers to an mRNA sequence or a portion thereof that directly codes for a polypeptide or its precursor.

The term “promoter element” or “promoter” refers to a DNA regulatory region capable of binding an RNA polymerase in a cell (e.g., directly or through other promoter-bound proteins or substances) and initiating transcription of a coding sequence.

The term “promoter sequence” is a region that is in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences.

The term “vector” refers to a nucleic acid assembly capable of transferring gene sequences to target cells (e.g., viral vectors, non-viral vectors, particulate carriers, and liposomes).

The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector.

The terms, “vector construct,” “expression vector,” and “gene transfer vector,” generally refer to any nucleic acid construct capable of directing the expression of a gene of interest and which can transfer gene sequences to target cells. Thus, the term includes cloning and expression vehicles, as well as viral vectors.

The term “antibody” refers to a whole antibody, both polyclonal and monoclonal, or a fragment thereof, for example a F(ab)₂, Fab, FV, VH or VK fragment, a single chain antibody, a multimeric monospecific antibody or fragment thereof, or a bi- or multi-specific antibody or fragment thereof. The term also includes humanized and chimeric antibodies.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient. In this instant, treatment involves use of this invention as a single delivery therapeutic, or multiple or repeated delivery therapeutic, or a control delivery therapeutic and is meant to be delivered locally, systemically, intravascularly, intramuscularly, intraperitoneally, inside the blood-brain barrier, or via other various routes.

The term “patient” refers to a biological system to which a treatment can be administered. A biological system can include, for example, an organ, a tissue, or a multi-cellular organism. A patient can refer to a human patient or a non-human patient.

Compositions.

In one embodiment, the step of increasing the amount of the amino acid sequence which is at least 70% identical to an amino acid sequence encoding an LMP protein or a fragment thereof is achieved by contacting the source cell with such amino acid sequence. A person of the ordinary skill in the art will appreciate that the amino acid sequence may be at least 70% identical to an amino acid sequence encoding an LMP protein or a fragment thereof, or at least 75% identical to an amino acid sequence encoding an LMP protein or a fragment thereof, or at least 80% identical to an amino acid sequence encoding an LMP protein or a fragment thereof, or at least 85% identical to an amino acid sequence encoding an LMP protein or a fragment thereof, or at least 90% identical to an amino acid sequence encoding an LMP protein or a fragment thereof, or at least 95% identical to an amino acid sequence encoding an LMP protein or a fragment thereof, or at least 99% identical to an amino acid sequence encoding an LMP protein or a fragment thereof, or 100% identical to an amino acid sequence encoding an LMP protein or a fragment thereof. Suitable non-limiting examples of the LMP proteins include an LMP-1 protein (SEQ. ID. NO. 1), an LMP-2 protein (SEQ. ID. NO. 2), an LMP-3 protein (SEQ. ID. NO. 3), and an LMP-1s protein (SEQ. ID. NO. 4). Accordingly, suitable non-limiting examples of the amino acid sequence of the instant invention comprise SEQ. ID. NO. 1, SEQ. ID. NO. 2, SEQ. ID. NO. 3, and/or SEQ. ID. NO. 4. The LMP proteins from species other than Homo Sapiens may also be used. Amino acid sequences of LMP proteins of other species are publicly available from different databases including, without limitations, Genbank. In another embodiment, the amino acid sequence of the instant invention comprises the fragment of the LMP protein, such as, for example, a LIM domain (comprising amino acids 400-452 of LMP-1 protein SEQ. ID. NO. 5), LIM motif (comprising amino acids 341-391 of LMP-1 protein, SEQ. ID. NO. 6) an osteogenic region (SEQ. ID. NO. 7, GAPPPADSAP) or a combination thereof. The exact amino acid sequence of the instant invention should ultimately be selected on the basis of the nature of the source cell, the nature of the target cell, and the nature of the desired change in phenotype of the target cell.

Optionally, the amino acid sequence of the instant invention further comprises a penetration means such as, for example, a TAT (e.g., SEQ. ID. NO. 8) or a PTD domain (including, without limitations, SEQ. ID. NO. 9-18). In another embodiment, the amino acid sequence of the instant invention may be packaged with a composition which advances the entry of the amino acid sequence of the instant invention into the source cell. For example, the amino acid sequence may be packaged with liposomes. See, e.g., U.S. Pat. Nos. 6,338,859, 5,631,018; 6,162,462; 6,475,779; 6,521,211; and 6,443,898, Felgner, J. H., et al. (1994). J. Biol. Chem. 269, 2550-2561. Zelphati, O., et al. (2001). J. Biol. Chem. 276, 35103-35110.

The kits and reagents (e.g., Pro-Ject Protein Transfection Reagent) for introducing the amino acid sequence of the instant invention may also be purchased from commercial suppliers, such as, for example, Pierce (Rockford, Ill., Catalog #89850).

In another embodiment, the step of increasing the amount of the amino acid sequence which is at least 70% identical to an amino acid sequence encoding an LMP protein or a fragment thereof is achieved by contacting the source cell with a nucleic acid encoding the amino acid sequence which is at least 70% identical to an amino acid sequence encoding an LMP protein or a fragment thereof. The nucleic acid sequences for the LMP-1, LMP-2, LMP-3, and LMP-1s proteins are known in the art. Sequences of other LMP proteins, including the LMP proteins from other species are also suitable for this invention and available from public and private sources, such as, for example, Genbank, and previously published patent and non-patent literature, including, without limitation, U.S. Pat. No. 6,300,127 (Hair) incorporated herein by reference to the extent it is not inconsistent with the instant disclosure. Optionally, the nucleic acid sequence may comprise an additional nucleic acid sequence encoding an amino acid sequence which would facilitate the translocation of the amino acid sequence of the instant invention into the nucleus of the source cell.

In one embodiment, the nucleic acid sequence of the instant invention is included within a vector by methods well known in the art utilizing endonuclease and ligase properties. The vector may be a plasmid or a virus. Suitable plasmid vectors include, without limitation, pUC18 and pUC19. Suitable viral vectors include adenoviral vectors, adeno-associated vectors and baculoviral vectors. Additional examples of vectors are listed in catalogs of different manufacturers, including, without limitation, Promega Corp. (Madison, Wis.), incorporated herein by reference in its entirety. Further, the vector may contain a promoter which directs the expression of the amino acid sequence of interest from the nucleic acid sequence. Suitable promoters include, without limitation, CMV, RSV, and TK. The vector containing the nucleic acid sequence encoding the amino acid sequence of interest is later introduced to the source cells.

A person of the ordinary skill in the art will undoubtedly appreciate that multiple methods exist for introducing the nucleic acid sequences into the source cells. Methods of introducing exogenous nucleic acid sequences are described in Sambrook and Russel, Molecular Cloning: A Laboratory Manual (3^{rd} Edition), Cold Spring Harbor Press, NY, 2000. These methods include, without limitation, physical transfer techniques, such as, for example, microinjection or electroporation; transfections, such as, for example, calcium phosphate transfections; membrane fusion transfer, using, for example, liposomes; and viral transfer, such as, for example, the transfer using DNA or retroviral vectors.

### Methods of Making Amino Acid and Nucleic Acid Sequences

A wide variety of techniques exists for manufacturing the amino acid sequences and the nucleic acid sequences of the instant invention.

In one embodiment, the amino acid sequences of the instant invention can be synthesized by standard solid peptide synthesis (Barany, G. and Merrifield, R. B., The Peptides 2:1 284, Gross, E. and Meienhofer, J., Eds., Academic Press, New York) using tert-butyloxycarbonyl amino acids and phenylacetamidomethyl resins (Mitchell, A. R. et al., J. Org. Chem. 43:2845 2852 (1978)) or 9-fluorenylmethyloxycarbonyl amino acids on a polyamide support (Dryland, A. and Sheppard, R. C., J. Chem. So. Perkin Trans. I, 125 137 (1986)). Alternatively, synthetic peptides can be prepared by pepscan synthesis (Geysen, H. M. et al., J. Immunol. Methods 03:259 (1987); Proc. Natl. Acad. Sci. USA 81:3998 (1984)), Cambridge Research Biochemicals, Cambridge, U.K. or by standard liquid phase peptide synthesis.

In another embodiment, the amino acid sequences may be purified from a cellular source. The suitable sources include cells which natively express peptides containing those sequences as well as an artificial expression system. The former include, without limitation, cultured osteoblasts. The purification techniques are well known in the art. One suitable method of purification is affinity chromatography. Essentially, in this technique, the cell extract is passed through a column impregnated with antibodies specifically recognizing the amino acid sequence of interest.

In yet another embodiment, the amino acid sequences of the instant invention can be recombinantly produced. The mRNA and cDNA sequences of the LMP proteins are well known in the art and available, for example, from Genbank. Thus, the primers may be designed to multiply the nucleic acid sequence encoding the amino acid sequence of interest by PCR (if the template is cDNA) or rtPCR (if the template is mRNA). Primer-directed amplification of DNA or cDNA is a common step in the expression of the genes of this invention. It is typically performed by the polymerase chain reaction (PCR). PCR is described in U.S. Pat. No. 4,800,159 to Mullis et al. and other published sources. The vector containing the nucleic acid sequence encoding the amino acid sequence of interest is later introduced to host cells.

The choice of the host cell system depends largely on the type of the vector and the type of the promoter. In general, the host cells include, without limitations, prokaryotic, yeast, insect, and mammal cells.

Further, depending on the type of the host cell, the codons of the nucleic acid sequences encoding the amino acid sequences of the instant invention can be selected for optimal expression in prokaryotic or eukaryotic systems. Host-vector systems include but are not limited to the following: bacteria transformed with bacteriophage DNA, plasmid DNA or cosmid DNA; microorganisms such as yeast containing yeast vectors; mammalian cell systems infected with virus (e.g., vaccinia virus, adenovirus, etc.); insect cell systems infected with virus (e.g., baculovirus). The expression elements of these vectors vary in their strength and specificities. Depending upon the host-vector system utilized, any one of a number of suitable transcription and translation elements can be used.

The amino acid sequences used in the kits and the methods of the instant invention can be purified or partially purified from cells comprising the vector, comprising the nucleic acid sequence encoding the amino acid sequence of interest, using known purification processes such as gel filtration and ion exchange chromatography. Purification may also include affinity chromatography with agents known to bind the respective amino acid sequences.

Further, the amino acid sequences of interest may be tagged, as described in more details below. In one non-limiting example, the recombinant nucleic acid sequences are fused with a nucleic acid sequence encoding glutathione-S-transferase (GST). The GST-tag is often used to separate and purify proteins that contain the GST-fusion. GST-fusion proteins can be produced in E. coli, as recombinant proteins. The GST part binds its substrate, glutathione. Sepharose beads can be coated with glutathione, and such glutathione-sepharose beads bind GST-proteins. These beads are then washed, to remove contaminating bacterial proteins. Adding free glutathione to beads that bind purified GST-proteins will release the GST-protein in solution.

Once purified, the cleavage of the amino acid sequences of the instant invention into fragments of amino acid residues can be achieved using proteolytic enzymes such as thrombin or clostridiopeptidase B (clostripain). The exact time required for proteolysis varies with each preparation and markedly depends upon the batch of clostripain used. Therefore, the optimum time for a single cleavage must be determined for each combination of clostripain batch and the amino acid sequence used. The protein fragments resulting from either thrombin or clostripain proteolysis may be further cleaved by digestion with trypsin, which cleaves on the carboxy terminus of lysine or arginine residues.

The sequence derived from proteolytic digestion may be identified using the Edman degradation method of protein sequencing. In addition, sequence analysis of the amino acid sequences of the instant invention may be accelerated by using an automated liquid phase amino acid sequenator, thereby allowing for the analysis of picomolar quantities of the recombinant proteins containing up to 50 amino acid residues in length.

Methods needed to construct and analyze the recombinant vectors (including the vectors for introduction into the source cell and the vectors for introduction into the host cell of the expression system) include, for example, restriction endonuclease digests and DNA sequencing. These techniques are well known in the art and have been described, for example, in Sambrook and Russel, Molecular Cloning: A Laboratory Manual (3 Edition).

### Cells

A variety of different cell types may be used as the source cells or the target cells. In general, the source cells and the target cells may belong to the same or to the different cell types. They may be undifferentiated, fully differentiated or de-differentiated. In different embodiments, the source and target cells may be independently selected from stem cells, kidney cells, neural cells (which, for the purposes of this application, includes, without limitations, different types of neurons, neuroprogenitor cells and glial cells), cardiac cells, smooth muscle cells, striated muscle cells, osteoblasts, osteoclasts, cartilage cells, endothelial cells, dental pulp cells, ligament cells, tendon cells, nucleus pulposus cells, annulus fibrosis cells, and any combination thereof. A person of ordinary skill in the art will recognize that in one embodiment, the change in a phenotype of a target cell (e.g., a stem cell) will lead to the differentiation of the target cell into a cell of an organ. Therefore, in one embodiment, the invention provides a method of generating an organ cell from a stem cell comprising (a) contacting a source cell with a polypeptide selected from the group consisting of an LMP-1 protein, an LMP-2 protein, an LMP-3 protein, an LMP-1s protein, SEQ ID NO: 5, SEQ ID NO: 6, SEQ ID NO: 7 or a combination thereof; (b) collecting a portion of the volume of the media of the source cell; and (c) contacting the stem cell with the portion of the volume of the media of the source cell. In different exemplary embodiments, the organ is a kidney, a nervous system, a heart, a smooth muscle, a striated muscle, a bone, a cartilage, a blood vessel, a tooth, a ligament, a tendon, or a disc.

In one embodiment, the source and/or target cells are bone marrow cells. These cells are readily available from an accessible source and can be harvested from human donors with minimal morbidity. If the bone marrow cells are used in the practice of the invention, the cell source may be whole bone marrow, concentrated bone marrow, filtered bone marrow, separated bone marrow cells, and cell populations isolated and culture-expanded from the bone marrow source. Notably, bone marrow contains a population of mesenchymal cells. It has been reported that transplanted human mesenchymal stem cells into cartilage might undergo site-specific differentiation into chondrocytes. See K. W. Liechty et al., Human Mesenchymal Stem Cells Engraft and Demonstrate Site-specific Differentiation After In Utero Transplantation in Sheep, 6 NAT. MED. 1282-6 (2000). Importantly, using human bone marrow cells obviates the practical problems of autologous or allogeneic disc cell harvest and greatly shortens the time required for cell preparation in the clinical transplantation procedure.

Adult bone marrow cells have been shown to differentiate into chondrocytes in vitro and in vivo. See D. J. Prockop, Marrow Stromal Cells as Stem Cells for Nonhematopoietic Tissues, 276 SCIENCE 71-4 (1997); M. F. Pittenger et al., Multilineage Potential of Adult Human Mesenchymal Stem Cells, 284 SCIENCE 143-7 (1999); P. Bianco et al., Bone Marrow Stromal Stem Cells: Nature, Biology, and Potential Applications, 19 STEM CELLS 180-92 (2001). Engineering of adult marrow cells to express chondrogenic genes has been reported to direct their differentiation towards cartilage in situ and hence to repair the cartilage. See N. Adachi et al., Muscle Derived, Cell Based Ex Vivo Gene Therapy for Treatment of Full Thickness Articular Cartilage Defects, 29 J. RHEUMATOL. 1920-30 (2002); Y. Gafni et al., Stem Cells as Vehicles for Orthopedic Gene Therapy, 11 GENE THER. 417-26 (2004). BMPs may promote osteogenic differentiation of mesenchymal stem cells, but due to the avascular and low oxygen tension environment within the disc, the mesenchymal stem cells are more likely to differentiate into chondrocytes. See D. A. Puleo, Dependence of Mesenchymal Cell Responses on Duration of Exposure to Bone Morphogenetic Protein-2 In Vitro, 173 J. CELL. PHYSIOL. 93-101 (1997); O. Fromigue et al., Bone Morphogenetic Protein-2 and Transforming Growth Factor-beta2 Interact to Modulate Human Bone Marrow Stromal Cell Proliferation and Differentiation, 68 J. CELL. BIOCHEM. 411-26 (1998); A. H. Reddi, Bone Morphogenetic Proteins, Bone Marrow Stromal Cells, and Mesenchymal Stem Cells: Maureen Owen Revisited, 1995 CLIN. ORTHOP. 115-9; M. K. Majumdar et al., BMP-2 and BMP-9 Promotes Chondrogenic Differentiation of Human Multipotential Mesenchymal Cells and Overcomes the Inhibitory Effect of IL-1, 189 J. CELL. PHYSIOL. 275-84 (2001).

Previous work has been performed to better discern the unique physiological role of different BMP signaling proteins, namely, by comparing the potency of BMP-6 with that of BMP-2 and BMP-4, for inducing rat calvarial osteoblast differentiation. Boden, et al., Endocrinology, 137, 3401 (1996). The study included an analysis of the process in first passage (secondary) cultures of fetal rat calvaria that require BMP or glucocorticoid for initiation of differentiation. In this model of membranous bone formation, glucocorticoid (GC) or a BMP will initiate differentiation to mineralized bone nodules capable of secreting osteocalcin, the osteoblast-specific protein. This secondary culture system is distinct from primary rat osteoblast cultures which undergo spontaneous differentiation. In this secondary system, glucocorticoid resulted in a ten-fold induction of BMP-6 mRNA and protein expression which was responsible for the enhancement of osteoblast differentiation. Boden, et al., Endocrinology, 138, 2920 (1997).

In another embodiment of this invention, cells other than bone marrow cells are useful, such as for example, different types of multipotential cells. The multipotential cells can be derived from various tissue sources in the body. In different embodiments, the cell population may be isolated from a living donor or a cadaver tissue source. Such tissue sources include, but are not limited to, adipose tissue, muscle tissue, peripheral blood, cord blood, blood vessels, skeletal muscle, skin, liver, and heart. In the practice of the invention, the cell source may include whole cells, concentrated cells, filtered cells, separated cells, and cell populations isolated and culture-expanded from a tissue source.

While in a preferred embodiment the target cells are derived from an autogeneic or, more preferably, an allogeneic source, the source cells may also be selected from a xenogeneic source. The xenogeneic source is preferably an animal which is closely related to humans, such as a primate, or more preferably, a member of family Hominidae, such as gorilla or chimpanzee. The choice of a non-human source for the source cell is advantageous because it is possible to produce a large quantity of the source cells of desired type from both embryos and adult animals without legal, ethical, economic, and other concerns accompanying the use of human embryos or adults as the source of the source cells.

After the source cells with an increased amount of the amino acid sequence which is at least 70% identical to an amino acid sequence encoding an LMP protein or a fragment thereof have been cultured for a sufficient time, which, dependent on a particular embodiment of the invention, may include as little as 5 minutes but as long as a few weeks, the volume of the culturing media or at least a portion thereof may be collected. In one embodiment, the culture time should not be long, so the method would be suitable for ex vivo intra-operative applications. In other embodiments, useful for ongoing culture harvest systems, the incubation time may be indefinite. Culture conditions also depend on the choice of the source cells and the target cells. In one embodiment, the culture conditions comprise 5% CO₂, 95% humidity. Optionally varying oxygen concentrations and/or mechanical forces may be used to enhance differentiation. If the source cells are grown in a monolayer, at least the portion of the volume of the culturing media may be collected by simple pooring or pipetting the media. If the source cells are grown in suspension, the step of collecting at least a portion of the volume of the media may be optionally combined with briefly centrifuging said at least the portion of the culture media remove the source cells. Further, the practitioner of the instant invention may choose to concentrate the culture media prior to contacting the target cells with at least the portion of the culture media. A person of the ordinary skill in the art will undoubtedly appreciate that equipment for concentrating the culture media is available. For example, Centricon concentrators (Millicore Corp., Billerica, Mass.) are suitable for concentrating the culture media. Different concentrators allow the practitioner to concentrate the culture media by 10, 50, or 100 or more times. In another embodiment, the media can be concentrated by centrifugation in vacuum.

Optionally, different analyses can be made to verify a presence or an amount of the different signalling molecules secreted by the source cells. As discussed above, these signalling molecules include, without limitations, BMP-2 and BMP-7 proteins. The suitable methods to analyze at least the portion of the volume of the media include, without limitations, radioimmunoassay, chromatography, and ELISA.

It is envisioned that upon being contacted with the at least the portion of the volume of the media, the target cell will develop into the desired cell type, such as for example, kidney cells, neural cells, cardiac cells, smooth muscle cells, striated muscle cells, osteoblasts, osteoclasts, nucleus pulposus cells, annulus fibrosis cells, chondroblasts, ligament cells, tendon cells, meniscus cells, or synovial cells. The development of the target cell may be verified by assessing a change in one or more of its characteristics. Such characteristics include, without limitations, changes in gene expression pattern, morphology, electrical activity, contractility, migration, attachment, or division rate. Depending on the type of the target cell, a person of the ordinary skill of the art may pick the characteristic to verify that the target cell reached or is on its way to reaching the desired phenotype. For example, neural cells have a specific electrical activity, morphology and markers (e.g., a microtubule-associated protein 2, β-III tubulin and NFM are examples of markers of neuronal differentiation; PSA-NCAM is a surface protein expressed on migratory neuroblasts, S100-β (15%) is the astrocyte-specific protein GFAP and vimentin are the astrocyte intermediate filament proteins. See Soltani et al, Am J Pathol. 2005 June; 166(6):1841-50; Deng et al, Stem Cells 2006 April; 24(4):1054-64). Kidney cells have specific morphology and express specific markers (e.g., nephrin), intervertebral disc cells express aminoglycans, proteoglycans (e.g., aggrecan, versican, lumican), and collagens, the presence of osteoblasts may be verified by bone nodules, calponin is expressed specifically in smooth muscle cells (Espinosa et al, Vet Pathol. 2002 March; 39(2):247-56), etc.

Further, the target cell may change its gene pattern expression. Preferably, the target cell expresses and secretes an increased amount of growth factors, such as, for example, BMP2 protein, a BMP4 protein, a BMP6 protein, a BMP7 protein, a BMP12 protein, a BMP13 protein, TGF-beta proteins, insulin growth factor proteins, and VEGF.

In another embodiment, the addition of said at least the portion of the volume of the culturing media may change the gene expression pattern of the target cell by decreasing or silencing genes which were expressed in the target cells prior to the addition of said at least the portion of the volume of the culturing media. In one embodiment, the addition of said at least the portion of the volume of the culturing media decreases expression of target gene which encodes a protein increasing catabolic activity of the target cell. Non-limiting examples of such proteins include groups of NF-kappa-B proteins, SMADs, ERKs, inflammatory cytokines, and any combination thereof.

The changes of the one or more characteristic of the target cell may be verified by a plurality of methods. For example, the change of morphology may be verified by microscopy. The changes in the division rate may be verified by [³H]-thymidine incorporation assay. The changes of the gene expression pattern may be verified on the level of the mRNA of the target gene by Northern blot or quantitative RT-PCR or real-time RT-PCR. The PCR-based techniques above may be performed by methods and kits available from commercial suppliers, including, without limitations, Ambion® (Austin, Tex.). The nucleic acid sequences for the target genes are available on Genbank, and the process of selection suitable primers and the hybridization probes are well within the skill of a person of the ordinary skill in the art. The changes on the protein level may be verified, for example, by immunocytochemistry or ELISA. The antibodies against these target genes may be obtained from commercial suppliers, such as, for example, RDI division of Fitzgerald Industries Intl. (Concord, Mass.), producing antibodies specific to individual BMP proteins, or Abcam® (Cambridge, UK).

In another embodiment, the antibodies to the target proteins or to the amino acid sequences of the instant invention may be created by the practitioners of the methods of the instant invention. For example, monoclonal antibodies can be produced by generation of hybridomas in accordance with known methods. Hybridomas formed in this manner are then screened using standard methods, such as ELISA, to identify one or more hybridomas that produce an antibody that specifically binds to the desired amino acid sequence or protein.

As an alternative to preparing monoclonal antibody-secreting hybridomas, a monoclonal antibody may be identified and isolated by screening a recombinant combinatorial immunoglobulin library (e.g., an antibody phage display library) to thereby isolate immunoglobulin library members that bind to the desired amino acid sequence or protein. Kits for generating and screening phage display libraries are commercially available from, e.g., Dyax Corp. (Cambridge, Mass.) and Maxim Biotech (South San Francisco, Calif.). Additionally, examples of methods and reagents particularly amenable for use in generating and screening antibody display libraries can be found in the literature.

Polyclonal sera and antibodies may be produced by immunizing a suitable subject, such as a rabbit, with the desired amino acid sequence or protein (preferably mammalian; more preferably human). The antibody titer in the immunized subject may be monitored over time by standard techniques, such as with ELISA, using immobilized marker protein. If desired, the antibody molecules directed against the desired amino acid sequence or protein may be isolated from the subject or culture media and further purified by well-known techniques, such as protein A chromatography, to obtain an IgG fraction, or by affinity chromatography, similar to methods described in Firestein et al., Neuron 24:659 (1999).

Fragments of antibodies to the desired amino acid sequence or protein may be produced by cleavage of the antibodies in accordance with methods well known in the art. For example, immunologically active F(ab′) and F(ab′)₂ fragments may be generated by treating the antibodies with an enzyme such as pepsin.

Thus, the invention provides for a method of targeted alteration of the phenotype of the target cell by contacting said target cell with at least a portion of the volume of the culture media from the source cells which have an increased amount of an amino acid sequence which is at least 70% identical to an amino acid sequence encoding an LMP protein or a fragment thereof. Accordingly, the methods of this invention may be useful for the use of the LMP proteins for decreasing tumor size or treating cancers; for influencing the behavior of neurologic tissues and cells for treatment of a stroke, Parkinson's Disease or Alzheimer's Disease; for influencing the behavior of the kidney; for providing treatment in cardiac therapy; for providing treatment in muscular therapy; and for providing treatment in vessel therapy.

In different embodiments of the invention, the target cell may be located either in vitro or in vivo (e.g., in the patient). Accordingly, if the target cell is located in vitro, the method of the instant invention comprises an additional step of delivering the target cell into the area of interest within the patient's body. If the target cell is located in vivo, then at least the portion of the volume of the media is delivered to the target cell.

In both scenarios, the at least the portion of the volume of the media may optionally be concentrated prior to contacting the target cell, as discussed above. In additional embodiments, the composition comprising of the volume of the culture medium and/or the target cells may optionally comprise at least one additional bioactive agent, including, without limitation, analgesics, anesthetics, antibiotics, anti-inflammatory compounds, radiocontrast media, and the like. Suitable non-limiting examples of useful bioactive agents are provided, for example, in U.S. patent application Ser. No. 11/705,942 (Marx et al), filed on Feb. 14, 2007.

Anti-inflammatory compounds include both steroidal and non-steroidal structures. Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting example of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14, 304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The variety of compounds encompassed by this group are well-known to those skilled in the art. For detailed disclosure of the chemical structure, synthesis, side effects, etc. of non-steroidal anti-inflammatory compounds, reference may be had to standard texts, including Rainsford, Anti-inflammatory and Anti-Rheumatic Drugs, Vol. I-III, CRC Press, Boca Raton, (1985), and Scherrer, et al., Anti-inflammatory Agents, Chemistry and Pharmacology 1, Academic Press, New York (1974), each incorporated herein by reference.

Mixtures of these non-steroidal anti-inflammatory compounds may also be employed, as well as the pharmacologically acceptable salts and esters of these compounds.

In addition, so-called “natural” anti-inflammatory compounds are useful in methods of the disclosed invention. Such compounds may suitably be obtained as an extract by suitable physical and/or chemical isolation from natural sources (e.g., plants, fungi, by-products of microorganisms). Suitable non-limiting examples of such compounds include candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, sea whip extract, compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Suitable analgesics include, without limitation, non-steroid anti-inflammatory drugs, non-limiting examples of which have been recited above. Further, analgesics also include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

Both the target cell (if located in vitro) and/or at least the portion of the volume of the culture medium (if the target cell is located in vivo) may be delivered to the target area by a plurality of methods. In general, the target cell (if located in vitro) and/or at least the portion of the volume of the culture medium may be administered via an intravenous injection, a direct injection into a location adjacent to the target cell or the target area, an intraperotoneal injection, a topical application to the target cell or the target area, an intradiscal injection, an intraarticular injection, an intraventricular injection, delivered with an implant (e.g., on the external and/or internal surfaces of the implant) or any combination thereof. For example, in one embodiment, the target cell and/or at least the portion of the volume of the culture medium may be delivered via a catheter placed within or adjacent (e.g., within 5 cm, or within 2 cm, or within 1 cm) to the target area. In one embodiment, the catheter may be semi-permanently positioned in the desired area, such that the practitioner will not need to remove the catheter between the administrations in the course of treatment. Yet, after the treatment has been accomplished, the catheter may be withdrawn from the patient's body. The catheter may optionally be connected to a reservoir containing either the at least the portion of the culture media or the target cells and further comprising a timer or any other mechanism to control the dose of the composition released into the target area within the patient's body. In another embodiment, the composition may be delivered by an injection from a syringe.

In another embodiment, the composition which comprises the at least the portion of the volume of the culture medium and, optionally, the target cell, may be incorporated within an implant, such as, for example, intervertebral disc implant, a nucleus pulposus implant, a heart implant, a brain implant, a vascular stent, a urethral stent, and any combination thereof. The carrier or the actual implant, in this invention, is an injectable, a solid, a moldable, or any structural, or other combination. It is made of natural polymers, synthetic polymers or a combination thereof, as described below.

In one embodiment, especially advantageous when the target cell is located in vivo, at least the portion of the volume of the culture medium, which, optionally, may be concentrated, as discussed above, may be incorporated simply by soaking the implant in the solution comprising said at least the portion of the volume of the culture medium. In another embodiment, the composition may be dripped, injected, sprayed, or brushed onto the implant. When placed into the target area (e.g., within the patient's body), the implant will release the composition, thus providing a sustained-release formulation. A person of ordinary skill in the art will appreciate that at this point, the culture media is essentially free of the LMP protein or fragment thereof or nucleic acid sequences encoding the LMP protein or the fragment thereof. Accordingly, this approach reduces or altogether eliminates the risks and drawbacks associated with traditional in vivo gene therapy, wherein the gene therapy agent is administered to the patient.

In another set of embodiments, the target cells are located in vitro. Upon being contacted with at least the portion of the volume of the culture medium, the target cells may then be cultured for an additional amount of time. Optionally, at this state, the cells may be sorted, for example, by using a cell sorter, to increase the relative ratio of the cells of the desired phenotype.

After the practitioner has a sufficient amount of the target cells, he can resuspend the target cells in a media and introduce the media comprising the target cells to the implant. In one embodiment, the media comprising the target cells may be dripped onto the implant while optionally being gently agitated to promote homogenous distribution of the target cells throughout the volume of the media. A person of the ordinary skill in the art will appreciate that other methods exist to incorporate the cells of the instant invention into suitable implants.

In different embodiments, the implant is made of natural polymers, synthetic polymers or a combination thereof. For example, the implant may be formed of polymers, such as synthetic biodegradable polymers, synthetic non-biodegradable polymers, natural polymers, or any combination thereof. The suitable non-limiting examples of synthetic biodegradable polymers include alpha-hydroxy acids, such as poly-lactic acid, polyglycolic acid, enantiomers thereof, co-polymers thereof, polyorthoesters, and combinations thereof.

The suitable non-limiting examples of synthetic non-biodegradable polymers include hydrogels such as PVA, delrin, polyurethane, polyethylene, co-polymers thereof and any combinations thereof.

The natural polymers suitable for the implant include, without limitations, collagen, elastin, silk, hyaluronic acid, chytosan, and any combinations thereof.

For example, the implant may be formed of a solid material, including, without limitation, polymethylmethacrylate, silicones, polyurethanes, polyvinyl alcohol, polyamides, aromatic polyamide, polyethers, polyester liquid crystal polymers, ionomers, poly(ethylene-co-methacrylic) acids, polybutylene terephtalate (PBT), polycarbonates, polyaminocarbonates, lactic acid, glycolic acid, lactide-co-glycolides, anhydrides, orthoesters, caprolactone, epoxy, and any combinations thereof. Implants formed of these and other suitable solid materials may be used both to deliver the composition to the cell located in vivo and to deliver the cell with the increased amount of the desired amino acid sequence to the target area.

In other sets of embodiments, the implant comprises a liquid composition which solidifies in vivo e.g., upon injection into the patient's body. Suitable materials which solidify in vivo include, without limitation, polysaccharides, proteins, polyphosphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), sulphonated polymers, poly(N-vinyl-2-pyrrolidone), polyethylene glycol, polyethyleneoxide, poly(2-hydroxy ethyl methacrylate), copolymers of acrylates with N-vinyl pyrrolidone, N-vinyl lactams, and any combination thereof. Implants which solidify in vivo are suitable for the embodiments, wherein the composition is delivered to the cell located in vivo.

In another embodiment, the cells may be injected into the target area by any of the methods described above (e.g., an injection by a syringe or delivery via a catheter).

Solid rigid implants are especially suitable for bone applications. Implants which are more flexible and pliable are more suitable for use as stents or for application where rigid implants may damage the surrounding tissue (e.g., cardiac implant).

Every patent and non-patent publication cited in the instant disclosure is incorporated into the disclosure by reference to the same effect as if every publication is individually incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of changing a phenotype of a target cell comprising:
a) increasing an amount of an amino acid sequence in a source cell, wherein the source cell is located within a volume of a media and wherein the amino acid sequence is selected from the group consisting of an LMP-1 protein, an LMP-2 protein, an LMP-3 protein, an LMP-1s protein, SEQ ID NO: 5, SEQ ID NO: 6, SEQ ID NO: 7 or combination thereof;
b) collecting at least a portion of the volume of the media; and
c) contacting the target cell with at least the portion of the media.

2. The method of claim 1, wherein the source cell and the target cell belong to the same cell type.

3. The method of claim 1, wherein the source cell and the target cell belong to the same lineage.

4. The method of claim 1, wherein the target cell is selected from the group consisting of kidney cells, neural cells, cardiac cells, smooth muscle cells, striated muscle cells, osteoblasts, osteoclasts, nucleus pulposus cells, annulus fibrosis cells, cartilage cells, endothelial cells, dental pulp cells, ligament cells, tendon cells, and any combination thereof.

5. The method of claim 1, wherein the target cell is not fully differentiated or de-differentiated.

6. The method of claim 1, wherein the target cell changes at least one of its morphology, electrical activity, contractility, migration, attachment, or division rate after being contacted with at least the portion of the volume of the media.

7. The method of claim 1, wherein the target cell changes its gene expression pattern after being contacted with at least the portion of the volume of the media.

8. The method of claim 1, wherein contacting the target cell with at least the portion of the volume of the media results in a repression of expression of a target gene.

9. The method of claim 8, wherein
the target cell is selected from osteoblasts, chondroblasts, nucleus pulposus cells, annulus fibrosis cells, ligament cells, tendon cells, meniscus cells, synovial cells, stem cells, cartilage cells, endothelial cells, dental pulp cells, ligament cells, tendon cells, neural cells and any combination thereof; and
the target gene encodes a protein increasing catabolic activity of the target cell.

10. The method of claim 8, wherein the protein is selected from the group of NF-kappa-B proteins, SMADs, ERKs, inflammatory cytokines, and any combination thereof.

11. The method of claim 1, wherein the source cell is contacted with a composition comprising the amino acid sequence which is at least 70% identical to the amino acid sequence encoding the LMP protein or the fragment thereof.

12. The method of claim 1, wherein the target cell is located within a patient.

13. The method of claim 18, wherein the target cell is contacted with at least the portion of the volume of the media via an intravenous injection, a direct injection into a location adjacent to the target cell, an intraperotoneal injection, a topical application to the target cell, an intradiscal injection, an intraarticular injection, an intraventricular injection, or any combination thereof.

14. The method of claim 18, wherein at least the portion of the volume of the media is applied to an implant or a carrier, and further comprising positioning of the implant or the carrier in an area adjacent to the target cell.

15. The method of claim 20, wherein the implant or the carrier releases the at least the portion of the media over a sustained period of time.

16. The method of claim 20, wherein said implant or carrier comprises at least one member of the group consisting of natural polymers, synthetic polymers, or a combination thereof.

17. The method of claim 20, wherein said implant or carrier comprises at least one member of the group consisting of collagen, collagen-ceramic combination, BCP, DBM, PRP, MC, elastin, fibrin, silk, fibrin, hyaluronic acid, chitosan, PLA, PGA, PLGA, a Polyorthoester, polycaprolactone, polypropylene fumarate, polyvinyl alcohol, polyesters, polyethers, polyhydroxyls, hydrogels, or a combination thereof.

18. An implant or a carrier comprising at least the portion of the volume of the media of claim 1.

19. The method of claim 1, wherein at least one of the source cell or the target cell is a stem cell.

20. The method of claim 1, wherein the source cell does not natively express the LMP protein.

21. The method of claim 1, further comprising contacting the target cell with at least one bioactive agent.

22. A method of increasing the production of a target cell protein in a target cell comprising:
(a) contacting a source cell with a polypeptide selected from the group consisting of an LMP-1 protein, an LMP-2 protein, an LMP-3 protein, an LMP-1s protein, SEQ ID NO: 5, SEQ ID NO: 6, SEQ ID NO: 7 or a combination thereof
(b) collecting a portion of the volume of the media of the source cell; and
(c) contacting the target cell with the portion of the volume of the media of the source cell, wherein said target cell protein is selected from the group consisting of a kidney cell specific protein, a neural cell specific protein, a cardiac cell specific protein, a smooth muscle cell specific protein, a striated muscle cell specific protein, an osteoblast specific protein, an osteoclast specific protein, a cartilage cell specific protein, an endothelial cell specific protein, a dental pulp cell specific protein, a ligament cell specific protein, a tendon cells specific protein, a nucleus pulposus cell specific protein, and an annulus fibrosis cell specific protein.

23. The method of claim 22, wherein
the target cell is selected from osteoblasts, chondroblasts, nucleus pulposus cells, annulus fibrosis cells, ligament cells, tendon cells, meniscus cells, synovial cells, stem cells, and any combination thereof; and
the target cell protein is selected from the group consisting of growth factors, aminoglycans, proteoglycans, a type I collagen protein, a type II collagen protein, a type III collagen protein, and any combination thereof.

24. The method of claim 23, wherein the growth factor is selected from the group consisting of a BMP2 protein, a BMP4 protein, a BMP6 protein, a BMP7 protein, a BMP12 protein, a BMP13 protein, TGF-beta proteins, insulin growth factor proteins, VEGF and any combinations thereof.
